SEQUENCE LISTING (63 sequences)

SEQ ID NO: 1 - protein, 84 aa, Oryctolagus cuniculus
QSVEESGGRLVTPGTPLTLTCTVSGFSLSWVRQAPGKGLEWIGRFTISKTSTTVDLKITSPTTEDTATYFCARWGTGTLVTISS

SEQ ID NO: 2 - protein, 86 aa, Oryctolagus cuniculus
QSVKESEGGLFKPTDTLTLTCTVSGFSLSWVRQAPGNGLEWIGRSTITRNTNLNTVTLKMTSLTAADTATYFCARWGQGTLVTVSS

SEQ ID NO: 3 - protein, 85 aa, Oryctolagus cuniculus
QSLEESGGDLVKPGASLTLTCTASGFSFSWVRQAPGKGLEWIARFTISKTSSTTVTLQMTSLTAADTATYFCARWGPGTLVTVSS

SEQ ID NO: 4 - protein, 87 aa, Homo sapiens
EVQLLESGGGLVQPGGSLRLSCAASGFTFSWVRQAPGKGLEWVSRFTISRDNSKNTLYLQMNSLRAEDTAVYYCAKWGRGTLVTVSS

SEQ ID NO: 5 - protein, 87 aa, Homo sapiens
EVQLVETGGGLIQPGGSLRLSCAASGFTVSWVRQAPGKGLEWVSRFTISRDNSKNTLYLQMNSLRAEDTAVYYCARWGQGTMVTVSS

SEQ ID NO: 6 - protein, 87 aa, Homo sapiens
EVQLVESGGGLVQPGGSLRLSCAASGFTVSWVRQAPGKGLEWVSRFTISRDNSKNTLYLQMNSLRAEDTAVYYCARWGQGTTVTVSS

SEQ ID NO: 7 - protein, 87 aa, Mus musculus
QVQLKESGPGLVAPSQSLSITCTVSGFSLTWVRQPPGKGLEWLGRLSISKDNSKSQVFLKMNSLQTDDTAMYYCARWGQGTLVTVSA

SEQ ID NO: 8 - protein, 87 aa, Mus musculus
EVMLVESGGGLVKPGGSLKLSCAASGFTFSWVRQTPEKRLEWVARFTISRDNAKNNLYLQMSSLRSEDTALYYCARWGAGTTVTVSS

SEQ ID NO: 9 - protein, 87 aa, Mus musculus
EVKLVESGGGLVKPGGSLKLSCAASGFTFSWVRQSPEKRLEWVARFTISRDNAKNTLYLQMSSLKSEDTAMYYCTRWGQGTTLTVSS

SEQ ID NO: 10 - protein, 80 aa, Oryctolagus cuniculus
AYDMTQTPASVEVAVGGTVTIKCWYQQKPGQRPKLLIYGVSSRFKGSGSGTEFTLTISGVECADAATYYCFGGGTEVVVK

SEQ ID NO: 11 - protein, 80 aa, Oryctolagus cuniculus
DVVMTQTPASVSEPVGGTVTIKCWYQQKPGQPPKLLISGVSSRFKASRSGTEFTLTISDLECADAATYYCFGGGTKVVVE

SEQ ID NO: 12 - protein, 80 aa, Oryctolagus cuniculus
ALVMTQTPASVSAAVGGTVTIKCWYQQKPGQPPKLLIYGVPSRFKGSRSGTEYTLTISGVQREDAATYYCFGGGTELEIL

SEQ ID NO: 13 - protein, 80 aa, Oryctolagus cuniculus
EVVMTQTPASVEAAVGGTVTIKCWYQQKPGQRPNLLIYGVPSRFKGSRSGTEFTLTISGVQREDAATYYCFGTGTKVEIK

SEQ ID NO: 14 - protein, 80 aa, Homo sapiens
DIQMTQSPSSLSASVGDRVTITCWYQQKPGKAPKLLIYGVPSRFSGSGSGTDFTLTISSLQPEDFATYYCFGQGTKLEIK

SEQ ID NO: 15 - protein, 80 aa, Homo sapiens
DIQMTQSPSSVSASVGDRVTITCWYQQKPGKAPKLLIYGVPSRFSGSGSGTDFTLTISSLQPEDFATYYCFGPGTKVDIK

SEQ ID NO: 16 - protein, 80 aa, Homo sapiens
AIQMTQSPSSLSASVGDRVTITCWYQQKPGKAPKLLIYGVPSRFSGSGSGTDFTLTISSLQPEDFATYYCFGGGTKVEIK

SEQ ID NO: 17 - protein, 80 aa, Mus musculus
EIVMTQSPATLSVSPGERATLSCWYQQKPGQAPRLLIYGIPARFSGSGSGTEFTLTISSLQSEDFAVYYCFGQGTRLEIK

SEQ ID NO: 18 - protein, 80 aa, Mus musculus
DIQMNQSPSSLSASLGDTITITCWYQQKKGNIPKLLIYGVPSRFSGSGSGTGFTLTISSLQPEDIATYYCFGGGTKLEIK

SEQ ID NO: 19 - protein, 80 aa, Mus musculus
DIVMTQSPSSLSVSAGDKVTMSCWYQQKPWQPPKLLIYGVPDRFTGSGSGTDFTLTISSVQAEDLAVYYCFGSGTKLEIK

SEQ ID NO: 20 - protein, 80 aa, Mus musculus
DIQMTQSPASLSASVGETVTITCWYQQKQGKSPQLLVYGVPSRFSGSGSGTQYSLKINSLQPEDFGSYYCFSDGTRLEIK

SEQ ID NO: 21 - protein, 80 aa, Mus musculus
SIVMTQTPKFLPVSAGDRVTMTCWYQQKPGQSPKLLIYGVPDRFTGSGSGTDFTFTISSVQVEDLAVYFCFGAGTKLELK

SEQ ID NO: 22 - protein, 79 aa, Oryctolagus cuniculus
QPVLTQSPSAAAALGASAKLTCWYQHQKGEAPRYLDGGVPDRFSGSSSGADRYLIISSVQADDEADYYCFGGGTQLTVT

SEQ ID NO: 23 - protein, 79 aa, Oryctolagus cuniculus
QPVLTQSPSVSAALGASARLTCWYQQQQGEAPRYLDGGVPDRFSGSSSGADRYLIIPSVQADDEADYYCFGGGTQLTVT

SEQ ID NO: 24 - protein, 79 aa, Homo sapiens
QPVLTQSSSASASLGSSVKLTCWHQQQPGKAPRYLMKGVPDRFSGSSSGADRYLTISNLQLEDEADYYCFGGGTKLTVL

SEQ ID NO: 25 - protein, 79 aa, Homo sapiens
QLVLTQSPSASASLGASVKLTCWHQQQPEKGPRYLMKGIPDRFSGSSSGAERYLTISSLQSEDEADYYCFGTGTKVTVL

SEQ ID NO: 26 - protein, 79 aa, Mus musculus
QLVLTQSSSASFSLGASAKLTCWYQQQPLKPPKYVMEGIPDRFSGSSSGADRYLSISNIQPEDEAIYICFGGGTKVTVL

SEQ ID NO: 27 - protein, 15 aa, Homo sapiens
MGWSCIILFLVATAT

SEQ ID NO: 28 - DNA, 24 bp, Artificial Sequence
  Oligo primer
tcgcactcaacacagacgctcacc

SEQ ID NO: 29 - DNA, 24 bp, Artificial Sequence
  Oligo primer
atggagactgggctgcgctggctt

SEQ ID NO: 30 - DNA, 25 bp, Artificial Sequence
  Oligo primer
gctcagcgagtagaggcctgaggac

SEQ ID NO: 31 - DNA, 25 bp, Artificial Sequence
  Oligo primer
ttggggggaagatgaagacagacgg

SEQ ID NO: 32 - DNA, 24 bp, Artificial Sequence
  Oligo primer
cagtgcaggcaggacccagcatgg

SEQ ID NO: 33 - DNA, 23 bp, Artificial Sequence
  Oligo primer
gccctggcaggcgtctcrctcta

SEQ ID NO: 34 - protein, 113 aa, Oryctolagus cuniculus
DIVMTQTPSSVSAAVGGTVTIKCQASDNIYSLLAWYQQKPGQPPKLLIYYTSDLTSGVPSRFSGSGYGTEFTLTISDLECADAATYYCQSYHYSKSSTYVNVFGGGTEVVVKG

SEQ ID NO: 35 - protein, 121 aa, Oryctolagus cuniculus
QSLEESGGGLVKPGASLALTCKASGFSFSLSFYMCWVRQAPGKGLEWIACIYSGSSGSTYYASWAKGRFTISKTSATTVTLQMTTLTAADTATYFCARSASSTTFHYFNLWGQGTLVTVSS

SEQ ID NO: 36 - protein, 87 aa, Mus musculus
EVKLQESGGGLVQPGGSLKLSCATSGFTFSWVRQTPEKRLEWVARFTISRDNAKNTLYLQMSRLKSEDTAMYYCARWGQGTTVTVSS

SEQ ID NO: 37 - protein, 87 aa, Homo sapiens
EVQLVESGGGLVQPGGSLRLSCAASGFTVSWVRQAPGKGLEWVSRFTISRDNSKNTLYLQMNSLRAEDTAVYYCARWGQGTLVTVSS

SEQ ID NO: 38 - protein, 85 aa, Oryctolagus cuniculus
QSLEESGGGLVKPGASLALTCKASGFSFSWVRQAPGKGLEWIARFTISKTSATTVTLQMTTLTAADTATYFCARWGQGTLVTVSS

SEQ ID NO: 39 - protein, 84 aa, Oryctolagus cuniculus; identical to SEQ ID NO: 1

SEQ ID NO: 40 - protein, 86 aa, Oryctolagus cuniculus; identical to SEQ ID NO: 2

SEQ ID NO: 41 - protein, 85 aa, Oryctolagus cuniculus; identical to SEQ ID NO: 3

SEQ ID NO: 42 - protein, 87 aa, Homo sapiens; identical to SEQ ID NO: 4

SEQ ID NO: 43 - protein, 87 aa, Homo sapiens; identical to SEQ ID NO: 5

SEQ ID NO: 44 - protein, 87 aa, Homo sapiens; identical to SEQ ID NO: 6

SEQ ID NO: 45 - protein, 87 aa, Mus musculus; identical to SEQ ID NO: 7

SEQ ID NO: 46 - protein, 87 aa, Mus musculus; identical to SEQ ID NO: 8

SEQ ID NO: 47 - protein, 87 aa, Mus musculus; identical to SEQ ID NO: 9

SEQ ID NO: 48 - protein, 80 aa, Mus musculus
DIVLTQSPSSLSASLGDTITITCWYQQKPGNIPKLLIYGVPSRFSGSGSGTGFTLTISSLQPEDIATYYCFGGGTKLEIK

SEQ ID NO: 49 - protein, 80 aa, Homo sapiens
DIQMTQSPSTLSASVGDRVTITCWYQQKPGKAPKLLIYGVPSRFSGSGSGTEFTLTISSLQPDDFATYYCFGGGTKVEIK

SEQ ID NO: 50 - protein, 80 aa, Oryctolagus cuniculus
DIVMTQTPSSVSAAVGGTVTIKCWYQQKPGQPPKLLIYGVPSRFSGSGYGTEFTLTISDLECADAATYYCFGGGTEVVVK

SEQ ID NO: 51 - protein, 80 aa, Oryctolagus cuniculus; identical to SEQ ID NO: 10

SEQ ID NO: 52 - protein, 80 aa, Oryctolagus cuniculus; identical to SEQ ID NO: 11

SEQ ID NO: 53 - protein, 80 aa, Oryctolagus cuniculus; identical to SEQ ID NO: 12

SEQ ID NO: 54 - protein, 80 aa, Oryctolagus cuniculus; identical to SEQ ID NO: 13

SEQ ID NO: 55 - protein, 80 aa, Homo sapiens; identical to SEQ ID NO: 14

SEQ ID NO: 56 - protein, 80 aa, Homo sapiens; identical to SEQ ID NO: 15

SEQ ID NO: 57 - protein, 80 aa, Homo sapiens; identical to SEQ ID NO: 16

SEQ ID NO: 58 - protein, 80 aa, Homo sapiens; identical to SEQ ID NO: 17

SEQ ID NO: 59 - protein, 80 aa, Mus musculus; identical to SEQ ID NO: 18

SEQ ID NO: 60 - protein, 80 aa, Mus musculus; identical to SEQ ID NO: 19

SEQ ID NO: 61 - protein, 80 aa, Mus musculus; identical to SEQ ID NO: 20

SEQ ID NO: 62 - protein, 80 aa, Mus musculus; identical to SEQ ID NO: 21

SEQ ID NO: 63 - protein, 5 aa, Artificial Sequence
  Synthetic oligopeptide
GGXGG

(19) United States
(12) Patent Application Publication
(10) Pub. No.: US 2004/0086979 A1
(43) Pub. Date: May 6, 2004

(54) Humanized rabbit antibodies

(76) Inventors: Dongxiao Zhang, Moraga, CA (US); Guoliang Yu, Berkeley, CA (US); Robert Pytela, San Francisco, CA (US); Fernando Jose Rebelo Do Couto, Pleasanton, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/638,210

(22) Filed: Aug. 7, 2003

Related U.S. Application Data
(60) Provisional application No. 60/404,117, filed on Aug. 15, 2002.

(51) Int. Cl. C12P 21/04; C07K 16/44

(52) U.S. Cl. 435/70.21; 530/387.3

(57) ABSTRACT

The invention provides methods for producing a modified nucleic acid that encodes a modified a rabbit antibody so that the modified rabbit antibody is less immunogenic in a non-rabbit host than an unmodified parent rabbit antibody. The invention further provides modified nucleic acids made by these methods, as well as vectors and host cells comprising the nucleic acids, and methods for producing the encoded modified antibodies. Also provided are modified rabbit antibodies encoded by subject nucleic acids, and compositions containing the same. The invention further provides kits for carrying out the subject methods.

### CROSS-REFERENCE

This application claims priority to U.S. provisional patent application serial No. 60/404,117, filed Aug. 15, 2002, which application is incorporated by reference herein in its entirety.

### INTRODUCTION

1. Field of the Invention

The field of this invention is antibodies, particularly methods of making rabbit antibodies that have reduced immunogenicity in non-rabbit hosts, such as humans and mice.

2. Background of the Invention

The rabbit immune system is fundamentally different from that of mouse. For example, the genome of mouse is estimated to have several hundred multi-family variable segments for heavy chain genes (V_{H}) which are primarily used to generate a large amount of the primary antibody repertoire through combinatorial joining of segments with D and/or J gene segments. The resulting VJ and VDJ gene rearrangements are then diversified by somatic diversification to develop the second antibody repertoire. Rabbits are quite different from mice in that although they have multiple germline V_{H} genes they actually use only one of them, V_{H}1, in most B lymphocytes (Knight and Becker, Cell 60: 963-970, 1990). In contrast to the human and mouse immune systems, combinational joining of multiple VH, D and JH gene segments contributes relatively little to the generation of antibody diversity and the initial antibody repertoire of rabbits is therefore rather limited. In fact, 80-90% of all variable domains of heavy chain immunoglobulin molecules in rabbits are encoded by V_{H}1. The existence of three major VH1 allotypes (alleles) of V_{H}1, V_{H}1-a1, V_{H}1-a2 and V_{H}1-a3, each with a related, but different, sequence, can introduce some sequence variability at the VH1-D-J recombination event. Despite the apparent lack of V_{H} segment variation, antisera generated in rabbits generally contain antibodies with a higher affinity that recognize a greater variety of epitopes than antisera generated in mice for many antigens (e.g. Krause et al, Adv Immunol 12: 1-56 (1970); Norrby et al, 1987 Proc. Natl. Acad. Sci.;84:6572-6 (1987); Raybould et al, Science 240:1788-90 (1988); Bystryn et al, Hybridoma 1: 465-72 (1982); Weller et al, Development. 100: 351-63 (1987)). While it is unknown exactly how this phenomenon is achieved, it is thought much of the antibody diversity in rabbits is generated almost purely by somatic gene conversion of rearranged VDJ genes (Becker and Knight, Cell 63:987-997, 1990), in contrast to the mechanism found in mice.

The introduction of non-human antibodies into humans usually results in the production of a specific immune response resulting from the presence of a foreign protein in the human body. In order to decrease these responses, efforts have been made to replace as much as possible of the original murine sequences with human counterparts, using recombinant DNA technology. Towards this end chimeric antibodies contain human antibody light chain and heavy chain constant domains that are joined to mouse antibody variable light chain and heavy chain domains. Chimeric antibodies still contain a large number of non-human amino acid sequences in the variable regions and, as such, a significant immune response may be mounted against such antibodies. CDR grafting is a humanization technique by which the antigen binding portions or complementarity determining regions (CDRs) of mouse monoclonal antibodies are grafted by recombinant DNA technologies into the DNA sequences encoding the framework (i.e. the non-CDR region) of human antibody heavy and light chains. One technical problem of CDR grafted antibodies is that usually they show considerable decreased affinity. To restore the original affinity certain original key framework residues, that are most likely involved in determining the conformation of the CDRs, must be reintroduced. Using a humanization different approach Roguska et al devised a “resurfacing” strategy for mouse antibodies where only exposed residues that are different to exposed residues of a human antibody are substituted.

There is an ongoing need for improved methods for making non-human antibodies, particularly rabbit antibodies, that are less immunogenic in humans and other mammalian hosts. The present invention addresses this, and other, needs.

Literature

References of interest include: U.S. Pat. Nos. 6,331,415 B1, 5,225,539, 6,342,587, 4,816,567, 5,639,641, 6,180,370, 5,693,762, 4,816,397, 5,693,761, 5,530,101, 5,585,089, 6,329,551, and publications Morea et al., Methods 20: 267-279 (2000), Ann. Allergy Asthma Immunol. 81:105-119 (1998), Rader et al,. J. Biol. Chem. 276:13668-13676 (2000), Steinberger et al., J. Bio. Chem. 275: 36073-36078 (2000), Roguska et al., Proc. Natl. Acad. Sci. 91: 969-973 (1994), Delagrave et al., Prot. Eng. 12: 357-362 (1999), Rogusca et al., Prot. Eng. 9: 895-904 (1996), Knight and Becker, Cell 60: 963-970 (1990) and Becker and Knight, Cell 63:987-997 (1990).

### SUMMARY OF THE INVENTION

The invention provides methods for producing a modified nucleic acid that encodes a modified a rabbit antibody so that the modified rabbit antibody is less immunogenic in a non-rabbit host than an unmodified parent rabbit antibody. The invention further provides modified nucleic acids made by these methods, as well as vectors and host cells comprising the nucleic acids, and methods for producing the encoded modified antibodies. Also provided are modified rabbit antibodies encoded by subject nucleic acids, and compositions containing the same. The invention further provides kits for carrying out the subject methods.

The subject methods involve substituting at least one nucleotide of a nucleotide sequence encoding an amino acid residue of a framework sequence of a rabbit antibody with at least one nucleotide of a nucleotide sequence encoding an amino acid residue of a non-rabbit host antibody.

In some embodiments, the non-rabbit host is a human, whereas in others the non-rabbit host is a mouse. In many embodiments, an addition and/or deletion of a residue of a rabbit framework sequence is also made. The subject antibodies, nucleic acid compositions and kits find use in a variety of applications, including diagnostics and therapeutic treatment and research of conditions and diseases, such as cancer.

In some embodiments, antibody variable regions are modified so that their surface is similar to the surface of non-rabbit host antibody variable regions without significantly altering the original binding properties. In general, the CDRs, the buried residues, and the residues that contact the CDRs are left unchanged during the modification process. This minimizes recognition by non-rabbit host antibodies because the surface of the modified antibody framework domains resemble a non-rabbit antibody. In many embodiments, providing that a search has been performed to identify the a similar human antibody, few residues are changed in this process. Nevertheless, these changes are likely to be very important to minimize the immunogenicity because they are done on the hydrophilic protein surface.

One advantage of the invention is that the methods provide a system for reproducibly and systematically humanizing or murinizing a rabbit monoclonal antibody, allowing a modified rabbit antibody to be used in a human or mouse host without generating a significant immune response to the antibody

Another advantage of the invention is that modified rabbit antibodies are typically of a higher affinity than mouse antibodies, increasing their therapeutic value.

These and other advantages and features of the invention will become apparent to those persons skilled in the art upon reading the details of the invention as more fully described below.

### BRIEF DESCRIPTION OF THE DRAWINGS

FIG. 1A shows a multiple sequence alignment of rabbit, human, and murine variable region frameworks. From top to bottom, the sequence are listed in the sequence listing as SEQ ID NOS:1-9.

FIG. 1B shows a multiple sequence alignment of rabbit, human, and murine variable region frameworks. From top to bottom, the sequence are listed in the sequence listing as SEQ ID NOS:10-26.

FIG. 2 shows a flow chart of an embodiment of the instant method: an algorithm for the humanization of rabbit antibodies. If one wanted to make a rabbit antibody less immunogenic in mice, or any other mammal, one would apply the same algorithm with the modification that antibody variable regions from that mammal would be used in step 3.

FIG. 3A, FIG. 3B, FIG. 3C and FIG. 3D show relative surface accessibility calculations for several high resolution structures as well as for a model of a rabbit IgG1 Kappa antibody (B1).

FIG. 4 shows a multiple sequence alignment of rabbit antibody sequences and similar non-rabbit sequences. From top to bottom, the sequences are listed in the sequence listing as SEQ ID NOS:36-62.

### DEFINITIONS

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a framework region” includes reference to one or more framework regions and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “host organism” means any animal that produces antibodies that have a variable regions that is structurally similar to those of rabbits. Exemplary host organisms include humans, a mice, rats, chickens, etc.

An amino acid residue that is in “close contact”, “close proximity” or “in close proximity to” another amino acid residue is an amino acid residue that is has a side chain that is close to, i.e., within 7, 6, 5 or 4 Angstroms of, a side chain of another amino acid. For example, an amino acid that are proximal to a CDR is a non-CDR amino acid that has a side chain that is close to a side chain of an amino acid in a CDR.

A “variable region” of a heavy or light antibody chain is an N-terminal mature domain of the chains. All domains, CDRs and residue numbers are assigned on the basis of sequence alignments and structural knowledge. Identification and numbering of framework residues is as described in by Chothia and others (Chothia Structural determinants in the sequences of immunoglobulin variable domain. J Mol Biol 1998;278:457-79).

VH is the variable domain of an antibody heavy chain. VL is the variable domain of an antibody light chain, which could be of the kappa (K) or of the lambda isotype. K-1 antibodies have the kappa-1 isotype whereas K-2 antibodies have the kappa-2 isotype and VL is the variable lambda light chain.

A “buried residue” is an amino acid residue whose side chain has less than 50% relative solvent accessibility, which is calculated as the percentage of the solvent accessibility relative to that of the same residue, X, placed in an extended GGXGG (SEQ ID NO:63) peptide. Methods for calculating solvent accessibility are well known in the art (Connolly 1983 J. appl. Crystallogr, 16, 548-558).

The terms “antibody” and “immunoglobulin” are used interchangeably herein. These terms are well understood by those in the field, and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N. Y., 2nd ed. (1984), and Hunkapiller and Hood, Nature, 323, 15-16 (1986),).

An immunoglobulin light or heavy chain variable region consists of a “framework” region interrupted by three hypervariable regions, also called “complementarity determining regions” or CDRs. The extent of the framework region and CDRs have been precisely defined (see, “Sequences of Proteins of Immunological Interest,” E. Kabat et al., U.S. Department of Health and Human Services, (1983)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

As used herein, the term “humanized antibody” or “humanized immunoglobulin” refers to an antibody comprising one or more CDRs from a rabbit antibody; and a rabbit framework region that contains amino acid substitutions and/or deletions and/or insertions that are based on a human antibody sequence. The rabbit immunoglobulin providing the CDRs is called the “parent” or “acceptor” and the human antibody providing the framework changes is called the “donor”. Constant regions need not be present, but if they are, they are usually substantially identical to human antibody constant regions, i.e., at least about 85-90%, preferably about 95% or more identical. Hence, in some embodiments, a full length humanized rabbit heavy or light chain immunoglobulin contains a human constant region, rabbit CDRs, and a substantially rabbit framework that has a number of “humanizing” amino acid substitutions, which will be described in detail below. In many embodiments, a “humanized antibody” is an antibody comprising a humanized variable light chain and/or a humanized variable heavy chain. For example, a humanized antibody would not encompass a typical chimeric antibody as defined above, e.g., because the entire variable region of a chimeric antibody is non-human. A modified antibody that has been “humanized” by the process of “humanization” binds to the same antigen as the parent antibody that provides the CDRs and is usually less immunogenic in humans, as compared to the parent antibody.

As used herein, the term “murinized antibody” or “murinized immunoglobulin” refers to an antibody comprising one or more CDRs from a rabbit antibody; and a rabbit framework region that contains amino acid substitutions and/or deletions and/or insertions that are based on a mouse antibody sequence. The rabbit immunoglobulin providing the CDRs is called the “parent” or “acceptor” and the mouse antibody providing the framework changes is called the “donor”. Constant regions need not be present, but if they are, they are usually substantially identical to mouse antibody constant regions, i.e., at least about 85-90%, preferably about 95% or more identical. Hence, in some embodiments, a full length murinized rabbit heavy or light chain immunoglobulin contains a mouse constant region, rabbit CDRs, and a substantially rabbit framework that has a number of “murinizing” amino acid substitutions, which will be described in detail below. In many embodiments, a “murinized antibody” is an antibody comprising a murinized variable light chain and/or a murinized variable heavy chain. For example, a murinized antibody would not encompass a typical chimeric antibody as defined above, e.g., because the entire variable region of a chimeric antibody is non-mouse. A modified antibody that has been “murinized” by the process of “murinization” binds to the same antigen as the parent antibody that provides the CDRs and is usually less immunogenic in mice, as compared to the parent antibody.

“Resurfacing” is the process by which a framework region residue at the surface of a rabbit antibody is altered, i.e. “resurfaced”, to make a rabbit antibody less immunogenic in a non-rabbit host. As such, “resurfacing” is a type of humanization strategy.

It is understood that the humanized antibodies designed and produced by the present method may have additional conservative amino acid substitutions which have substantially no effect on antigen binding or other antibody functions. By conservative substitutions is intended combinations such as gly, ala; val, ile, leu; asp, glu; asn, gln; ser, thr; lys, arg; and phe, tyr.

As used herein, the terms “determining,” “measuring,” and “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

As used herein the term “isolated,” when used in the context of an isolated antibody, refers to an antibody of interest that is at least 60% free, at least 75% free, at least 90% free, at least 95% free, at least 98% free, and even at least 99% free from other components with which the antibody is associated with prior to purification.

The terms “treatment” “treating” and the like are used herein to refer to any treatment of any disease or condition in a mammal, e.g. particularly a human or a mouse, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

### DETAILED DESCRIPTION OF PREFERRED EMBODIMENTS

The invention provides methods for producing a modified nucleic acid that encodes a modified a rabbit antibody so that the surface of the modified rabbit antibody is more similar to that of an antibody from a non-rabbit host and is thereby less immunogenic in the non-rabbit host than an unmodified parent rabbit antibody. The invention further provides modified nucleic acids made by these methods, as well as vectors and host cells comprising the nucleic acids, and methods for producing the encoded modified antibodies. Also provided are modified rabbit antibodies encoded by subject nucleic acids, and compositions containing the same. The invention further provides kits for carrying out the subject methods.

The subject antibodies, nucleic acid compositions and kits find use in a variety of applications, including diagnostics and therapeutic treatment and research of conditions and diseases, such as cancer.

In further describing the subject invention, methods of producing a modified nucleic acid that encodes a rabbit antibody with reduced immunogenicity in a non-rabbit host and, nucleic acids produced by the methods, as well as modified antibodies encoded by the modified nucleic acids, are described first followed by a review of the methods and representative applications in which the subject systems find use and kits that include the subject systems.

Methods of Resurfacing a Rabbit Antibody

The instant invention provides methods of producing a modified nucleic acid that comprises a nucleotide sequence encoding a modified rabbit antibody with a surface that is similar to that of a non-rabbit host antibody. These antibodies are usually less immunogenic in a non-rabbit host than an unmodified, parent rabbit antibody, while retaining specific binding to a predetermined antigen with high affinity. These methods produce nucleic acids containing nucleotide sequences that encode antibodies that have reduced immunogenicity in a non-rabbit host (e.g. a human or mouse), as compared to an unmodified parent rabbit antibody, and have binding affinities of at least about 10⁷ M⁻¹, 5×10⁷ M⁻¹, 10⁸ M⁻¹, or more usually 10⁹ M⁻¹ to 10¹⁰ M⁻¹, or higher to an antigen to which the unmodified parent antibody binds. The modified rabbit antibodies encoded by the modified nucleic acids have a rabbit framework sequence that is substituted by at least two contiguous or two discontiguous amino acids (i.e. separated by one or more amino acids) from a non-rabbit immunoglobulin heavy chain variable domain (V_{H}) or immunoglobulin light chain variable domain (V_{L}) (variable lambda or variable kappa) framework sequence. In most embodiments, the substituted amino acids are present on the surface of said non-rabbit antibody. The modified rabbit antibodies can be produced economically in large quantities and find use, for example, in the treatment and diagnosis of various human and mouse disorders by a variety of techniques.

Methods of Producing a Modified Nucleic Acid

In the methods of the invention, a nucleic acid encoding a modified rabbit antibody is made. In one embodiment, the method substitutes at least one nucleotide of a nucleotide sequence or codon encoding a V_{H} and/or V_{L} framework amino acid residue of a parent rabbit antibody with a nucleotide of a nucleotide sequence or codon encoding an amino acid residue from a non-rabbit V_{H} and/or V_{L} framework that shares a high degree of amino acid sequence identity. In many embodiments, where more than one amino acid is substituted, the substituted encoded amino acids are contiguous amino acids, which may encompass an entire framework region, whereas in other embodiments the substituted encoded amino acids are non-contiguous, i.e. the amino acids of a pair of substituted amino acids may be spaced by 1, 2, 3, 4, 5, 6, 7, 8, 9 or even 10 or more amino acids that are not substituted. In other embodiments, the substituted amino acids are a mixture of contiguous and non-contiguous amino acids, where 2, 3, 4 or 5 contiguous amino acids may be substituted, and 1, 2, 3, 4, or more than 5 non-contiguous amino acids may also be substituted.

In general, the method involves 1) identifying an amino acid of a framework region of a parent rabbit antibody that differs from an amino acid at a corresponding position of a non-rabbit antibody by comparing the amino acid sequence of the parent rabbit antibody framework region to the amino acid sequence of the non-rabbit antibody framework region; and (2) substituting at least one nucleotide of a nucleotide sequence encoding the identified amino acid, to form a modified rabbit nucleic acid sequence that encodes said corresponding amino acid. In most embodiments, the methods identify V_{H} and V_{L} chain framework amino acids that are on the surface of a rabbit antibody and exchanges nucleotides in nucleic acid sequences encoding those residues with nucleotides of nucleic acid sequences encoding amino acids at the equivalent position of non-rabbit V_{H} and V_{L} chain framework regions. Further details of these steps are provided below.

Rabbit Immunoglobulin V_{H} and V_{L} Chain Sequences

As a first step in the process, the amino acid sequence of a rabbit antibody framework region is compared with antibody framework regions of non-rabbit antibodies, which non-rabbit antibody framework regions share a high degree of amino acid sequence identity to the rabbit antibody framework. In some embodiments, the rabbit antibody is a known rabbit antibody. In other embodiments, the rabbit antibody is generated using known methods.

Rabbit antibodies are generated by immunizing a rabbit with an antigen or mixture of antigens. Rabbit immunoglobulin heavy and light chain variable domain framework sequences are usually identified by sequencing the nucleic acids (particularly cDNAs) that encode them. These nucleic acids may be isolated from any antibody-producing cell or mixture of cells e.g. bone marrow, spleen, etc., derived from an immunized rabbit. In most embodiments, antibody-encoding nucleic acids are isolated from these cells using standard molecular biology techniques such as polymerase chain reaction (PCR) or reverse transcription PCR (RT-PCR) (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

In many embodiments, however, rabbit antibody-encoding nucleic acids are isolated from a rabbit antibody-producing hybridoma cell. In order to produce rabbit antibody-producing hybridoma lines, rabbits are immunized with an antigen and once a specific immune response of the rabbit has been established, cells from the spleen of the immunized rabbit are fused with a plasmacytoma cell line such as 240E (Spieker-Polet et al, Proc. Natl. Acad. Sci. 92: 9348-9352, 1995). After fusion, the cells are grown in medium containing hypoxanthine, aminopterin, and thymidine (HAT) to select for hybridoma growth, and after 2-3 weeks, hybridoma colonies appear. Supernatants from these cultured hybridoma cells are screened for antibody secretion by enzyme-linked immunosorbent assay (ELISA) and positive clones secreting monoclonal antibodies specific for the antigen can be selected and expanded according to standard procedures (Harlow et al,. Antibodies: A Laboratory Manual, First Edition (1988) Cold spring Harbor, N.Y.; and Spieker-Polet et al., supra).

In other embodiments, the rabbit antibody-encoding nucleic acids are isolated from individual B-cells by isolating single cells by any known method. Exemplary methods include 1) performing flow cytometry of cell populations obtained from rabbit spleen, bone marrow, lymph node or other lymph organs followed by single-cell plating, e.g., through incubating the cells with labeled anti-rabbit IgG and sorting the labeled cells using a FACSVantage SE cell sorter (Becton-Dickinson, San Jose, Calif.); and 2) plating of plasma cells in multi-well plates at limiting dilutions. Cells can be directly sorted into 96-well or 384-well plates containing RT-PCR buffer, and subjected to RT-PCR with nested primers specific for the IgG heavy and light chains. As an alternative to cell sorting, limiting dilution cell plating can be used in order to obtain single B cells.

The methods of the invention, although appropriate for modifying any rabbit antibody, are usually used to modify a “natural” antibody, where the heavy and light immunoglobulins of the antibody have been naturally selected by the immune system of a multi-cellular organism, as opposed to unnaturally paired antibodies made by e.g. phage display. As such, the subject parental antibodies do not usually contain any viral (e.g., bacteriophage M13)-derived sequences.

The isolated rabbit nucleic acid encodes a framework region of a “parent” antibody.

Sequence Comparison

Once the rabbit immunoglobulin heavy and/or light chain variable domain framework amino acid sequences are determined, they are usually compared to a database of sequences of non-rabbit immunoglobulin chains in order to identify corresponding framework sequences of non-rabbit antibodies. Typically, one of the 10 most similar framework region sequences in terms of amino acid sequence identity (either by percent identity or P-value) to a parental framework sequence will be used as an amino acid residue donor. Usually, one of the three most similar framework region sequences in terms of amino acid sequence identity (percent identity or P-value) to a parent framework sequence will be used as an amino acid residue donor. The selected surface residue donor framework region will typically have at least about 55%, at least about 65% identity, at least about 75%, at least about 80%, at least about 85%, at least about 90%, or at least about 95% amino acid sequence identity in the framework region to the parent framework region. In many embodiments, a sequence containing a variable domain of a heavy or light chain immunoglobulin containing at least one framework region is compared to a database in order to identify similar sequences in the database. In some embodiments, sequences are compared to an amino acid sequence that is not stored in a database, e.g. to the sequence of a newly sequenced antibody.

In some embodiments, both the light and heavy chains from the same non-rabbit antibody may be used as surface residue donors.

Various antibody databases can be searched to identify the most homologous non-rabbit antibody immunoglobulins for a given rabbit immunoglobulin sequence. In addition to National Center for Biotechnology Information (NCBI) databases, several of the most commonly used databases are listed below:

V BASE—Database of Human Antibody Genes: This database is maintained by the medical research council (MRC), of Cambridge UK and is provided via the website: www.mrc-cpe.cam.ac.uk. This database is comprehensive directory of all human germline variable region sequences compiled from over a thousand published sequences, including those in the current releases of the Genbank and EMBL data libraries.

Kabat Database of Sequences of Proteins of Immunological Interest (Johnson, G and Wu, TT (2001) Kabat Database and its applications: future directions. Nucleic Acids Research, 29: 205-206) found at the website of Northwestern University, Chicago (immuno.bme.nwu.edu). The kabat database is also available at the nih/ncbi site

Immunogenetics Database: Maintained by and found at the website of the European Bioinformatics Institute: www.ebi.ac.uk. This database is integrated specialized database containing nucleotide sequence information of genes important in the function of the immune system. It collects and annotates sequences belonging to the immunnglobulin superfamily which are involved in immune recognition.

ABG: Germline gene directories of the mouse—a directory of mouse VH and VK germline segments, part of the webpage of the Antibody Group at the Instituto de Biotecnologia, UNAM (National Univfrsity of Mexico)

Built-in searching engines can be used to search for most similar sequences in terms of amino acid sequence homology. In the methods of this invention, BLAST (Altschul et al., J. Mol. Biol. 215:403-10, 1990) is performed using default parameters, including choosing the BLOSUM62 matrix, an expect threshold of 10, low complexity filter off, gaps allowed, and a word size of 3.

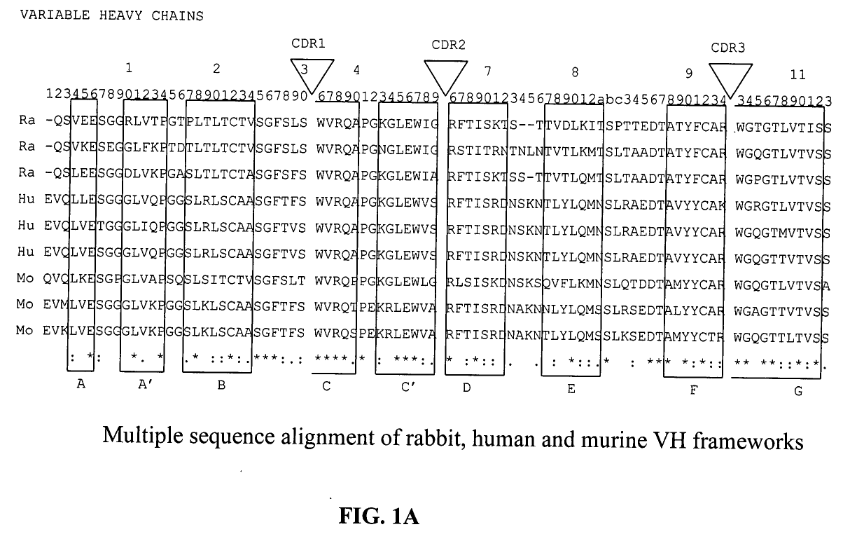

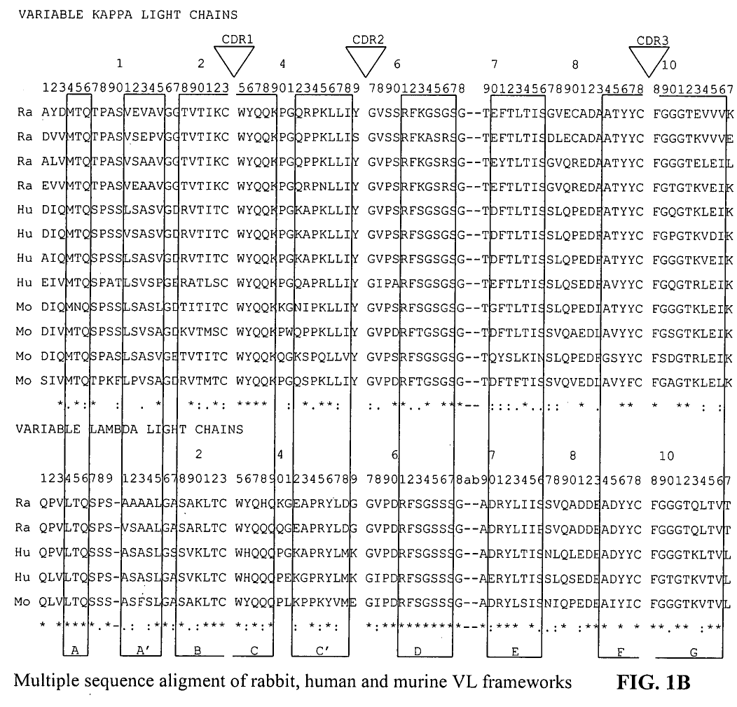

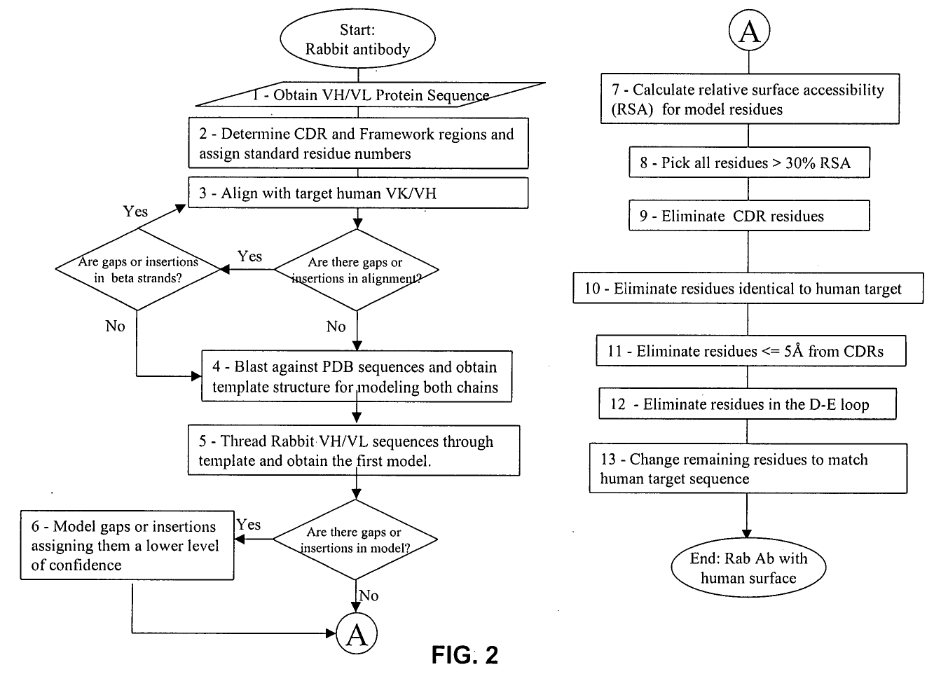

Rabbit immunoglobulin framework regions may be used to search for similar human or mouse immunoglobulin framework regions. BLAST search examples of rabbit V_{H}-1 genes are given in FIG. 1 and FIG. 2. The same search process can also be performed with emphasis on the homology for the solvent accessible residues.

Modifying the Nucleotide Sequence Encoding a Framework Region of a Rabbit Antibody

In many embodiments, nucleotides of nucleic acids encoding amino acid residues that are found on the surface of an antibody molecule are substituted. “Surface amino acid residues” are those that are solvent accessible in a mature antibody. “Surface amino acid residues” are also those that, by virtue of being solvent accessible, are more likely to be recognized by the immune system of a host as foreign, and therefore most likely to provoke an immune response in the host. Residues on the surface of a non-rabbit host “donor” antibody framework or a parent rabbit “acceptor” antibody framework are identified by comparing the sequence of the V_{H} or V_{L}-chain framework sequence of the antibody to the V_{H} or V_{L}-chain framework sequence of an antibody of known structure, or by molecular modeling. In many embodiments of the invention, a rabbit or non-rabbit framework sequence is aligned with a sequence of an antibody of known structure, and the rabbit framework residues corresponding to surface residues of the non-rabbit framework residues are identified.

Methods for aligning antibody sequences to sequences from antibodies of known structure have been described (Padlan et al Mol. Immunol. 28: 489-98 (1991); Pedersen J. Mol. Biol. 235: 959-73 (1994); Roguska et al Proc. Natl. Acad. Sci. USA 91: 969-73 (1994)). Usually residue is a surface residue if its relative accessibility is greater than 30% (Pederson et al, 1994, supra). Antibodies of known structure may be found in the following databases: 1) Antibodies-Structure and Sequence-database provides a query interface to the Kabat antibody sequence data, general information on antibodies and crystal structures and links to other antibody-related information, 2) the BMCD Biological Macromolecular Crystallization Database and the NASA Archive for Protein Crystal Growth Data (version 2.00), 3) Macromolecular Structure Database for Crystallographic Laboratories 4) PDB-Protein Data Bank at Brookhaven National Laboratory, an archive of experimentally determined three-dimensional structures of biological macromolecules.

The relative accessibility of amino acid residues can also be calculated using a method of DSSP (Dictionary of Secondary Structure in Proteins; Kabsch and Sander 1983 Biopolymers 22: 2577-637) and solvent accessible surface area of an amino acid may be calculated based on a 3-dimensional model of an antibody, using algorithms known in the art (e.g., Connolly, J. Appl. Cryst. 16, 548 (1983) and Lee and Richards, J. Mol. Biol. 55, 379 (1971), both of which are incorporated herein by reference).

In most embodiments of the invention, a parent nucleic acid is modified such that at least one amino acid of the framework of the encoded rabbit antibody is substituted with at least one amino acid at an equivalent position of a non-rabbit antibody. In many embodiments, the number of amino acids substituted is 2-100 or more, e.g. 2-5, 6-10, 11-15, 16-20, 21-40, 41-50, 51-60, 61-70, 71-80, 81-90, or 91-100 or more. The substituted amino acids may be in a heavy chain variable domain framework region, a light chain variable domain framework region, or both. In some embodiments the substituted amino acids are contiguous amino acids, where the length of a contiguous stretch of amino acids is an entire framework region sequence, or a contiguous subsequence thereof where the number of amino acids in the subsequence is 2-5, 6-10, 11-15 or 16-20 or more.

In most embodiments the substituted encoded amino acids are not contiguous, and may consist of a group of non-contiguous amino acids predicted to be on the surface of the parental or donor antibody. In these embodiments, an amino acid on the framework of the parental rabbit antibody is usually substituted by a corresponding amino acid on the non-rabbit donor antibody. In this respect “corresponding” means an amino acid residue on a donor sequence is positioned across from a residue on a parent sequence when the two sequences are aligned. Of course, as is known in the art (e.g. Roguska et al, P.N.A.S. 91: 969-973, 1994; Kabat 1991 Sequences of Proteins of Immunological Interest, DHHS, Washington, D.C.), sometimes one, two or three gaps and/or insertions of up to one, two, three or four codons should be made to one or both of the nucleic acids encoding the antibody framework sequence in order to accomplish an alignment. As such, in many embodiments, codons are inserted into or deleted from the parent rabbit nucleic acid in order to accomplish an alignment between the parent rabbit sequence and the non-rabbit sequence.

With Specific Reference to FIG. 2, the Subject Method is Described as Follows:

Steps 1, 2, 3

The protein sequence of the variable regions is deduced from their respective DNA sequences (step 1). The protein sequences are then analyzed and the positions of the CDRs are defined as described by Kabat, and residue numbers to the framework residues assigned (step 2). There are several ways accomplish step 2, and programs exist that assign residue numbers automatically. Some of these programs can be found on the Internet. However, the programs work better with murine and human antibody sequences than with rabbit antibody sequences. One can also perform a blast search of the Kabat database and then number the new rabbit antibody sequence as the Kabat sequences. In an alternative embodiment, step 2 may be done using a preexisting multiple sequence alignment between rabbit, human, and murine sequences such as the one shown in FIGS. 1A, 1B, and align the new sequence using the conserved residues as anchors. For example, kappa and lambda chains must have cysteine residues at positions 23 and 88, respectively. Other conserved framework residues have been described (Chothia C, Gelfand I, Kister A. Structural determinants in the sequences of immunoglobulin variable domain. J Mol Biol 1998 May 1;278(2):457-79). Once residue numbers have been assigned, the position of the beta strands will be known (see FIGS. 1A, 1B). One can then proceed to step 3 and find target host antibody sequences. This could be done by a blast search against the host's germline sequences or against all known host's antibody sequences. If there are multiple good choices for target host sequences one should pick the ones that are more commonly found in the host. For example human VH3 chains are found more frequently than human VH2 chains. One should then examine the alignment between the rabbit sequence and the target host sequence, all gaps and insertions should be in the CDRs or in regions outside the beta sheets. If one finds insertions or deletions in beta sheet regions most likely the alignment is incorrect. One can expect a one or two-residue deletion in the D-E loop of the rabbit VH, relative to the human VH, in many cases. In many embodiments, such a deletion indicates that the alignment is correct.

FIGS. 1A and 1B are multiple sequence alignments of rabbit, human, and murine variable region frameworks. CDR sequences were excluded in order to show the important information in a more compact fashion, but the CDR insertion points are indicated precisely. Frameworks (FR) are not indicated, but they are placed in sequence alternating with the CDRs as follows: FR1, CDR1, FR2, CDR3, FR3, CDR3, FR4. Beta strands are indicated (A, A′, B, C, C′, D, E, F, G). Beta strand C″ is not indicated because it is part of CDR2 which is not shown. Sequences in FR4 are not necessarily a continuation of the previous variable region sequences (FR1, FR2, FR3) because gerrnline V regions which encode the first frameworks and J-regions, which encode FR4 are also not contiguous. Standard Kabat sequence numbers are indicated on top. These numbers are important because they point to a structural position. Note that pdb structure files do not necessarily follow this convention. There are also other numbering systems. In principle any numbering system can be used as long as it is done consistently throughout the procedure, but the Kabat numbering system is the most generally accepted.

FIGS. 1A and 1B, among other things, demonstrate the homology between antibodies of different mammalian species. Second they make certain differences conspicuous, such as, for example, that rabbit VH chains can lack one or two residues from the D-E loop relative to most human and murine antibody chains. Third, they indicate precisely where the CDRs and the beta strands are, which is a requirement for modeling an antibody. The figures are also very helpful in obtaining alignments between the rabbit antibody chains and either the target host sequences or the sequences of the structural template used for modeling.

Step 4.

The rabbit sequence is now compared to (e.g., blasted against) the pdb database to find a suitable structure for performing the threading or homology modeling. Virtually, any structure of a protein belonging to the Ig superfamily would be useful but because there are hundreds of antibody structures available we can usually find structures of paired VH/VL chains whose protein sequences are very similar to those of the rabbit antibody. Naturally, the closest the similarities between sequences the better the resulting model will be.

Steps 5, 6.

There are several programs that can be used to build a model by homology. Some of these programs can be purchased but some are also available through the internet. For example, the Swiss Pdb Viewer, also known as “Deep View” can be used to model proteins by homology. If there are gaps or insertions in loops of the rabbit antibody relative to the loops of the template structure, those can be modeled using other structures. CDRs may be straightforward to model if they belong to a known canonical structure. This will almost certainly be always true for CDR L2, for example. However, it is frequently not possible to assign canonical structures to rabbit CDRs and it may be difficult to find good template structures to model them. In particular, one can expect great difficulty in finding good structural templates for CDRS L3 and H3. It may be difficult to find a good model for the D-E loop as well. However, the modeling of the CDR loops and of the D-E loop does not have to be perfect, because these loops are not changed by the method of this invention (steps 9, 10). In fact modeling of the CDR's and of the D-E loop is not absolutely required for this invention, as long as one knows from other antibody structures which surface residues are likely to contact CDRs. However, if CDR and D-E loop modeling is done to a reasonable degree of confidence, particularly in the regions adjacent to the frameworks, this will facilitate the model visualization and calculations in step 7. What matters is whether residue side chains are exposed to solvent or not. Solvent exposure is determined mostly by the particular position of a residue in the beta sheet and by the surrounding residues. In other words, a residue side chain has the freedom to rotate only from the beta carbon on. But the beta carbon's position itself is frozen in place as determined by the particular sequence position of its residue in the beta sheet. It cannot flip around and bury itself, for example. Therefore, an exposed residue will most likely always be detected as exposed regardless of the accuracy of the model as long as the residue number assignment is correct. Obviously, the same is not necessarily true for large loop regions because there are many possible conformations for the loop sequences. There are four loops on the “top” of each chain. Three of them are overlap with, or are the CDRs. The fourth is the D-E loop. While many times some of these loops cannot be accurately modeled for rabbit antibodies, none of these loops is changed according to this invention. One could change the D-E loop to match an often larger loop of human, or mouse, antibodies but this is a calculated risk. Residues in the D-E loop sometimes make contact with CDR residues, so, while one can certainly alter amino acids in this loop, one should not be surprised if in some cases the resulting modified antibody has lower affinity than the original antibody.

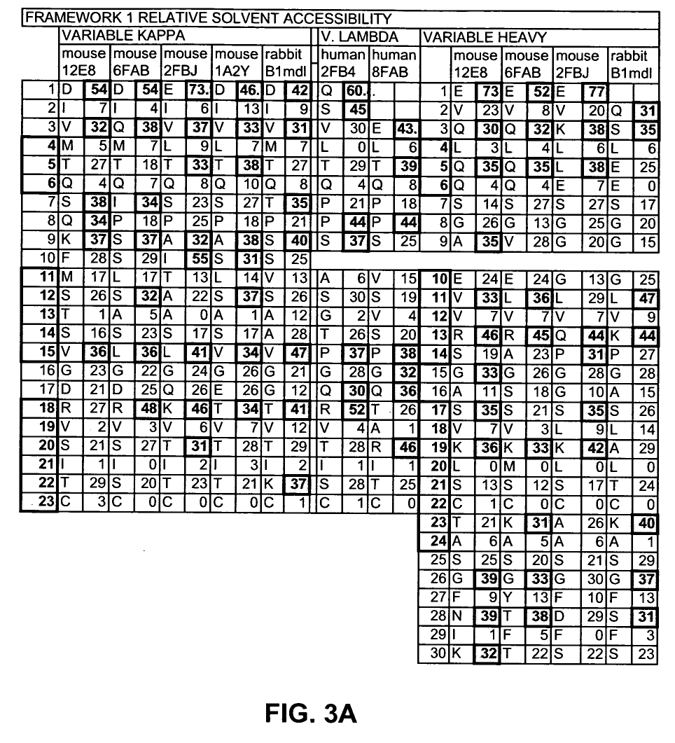

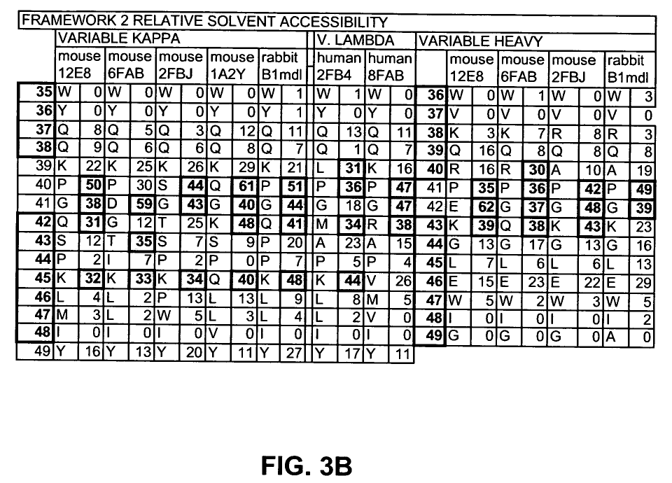

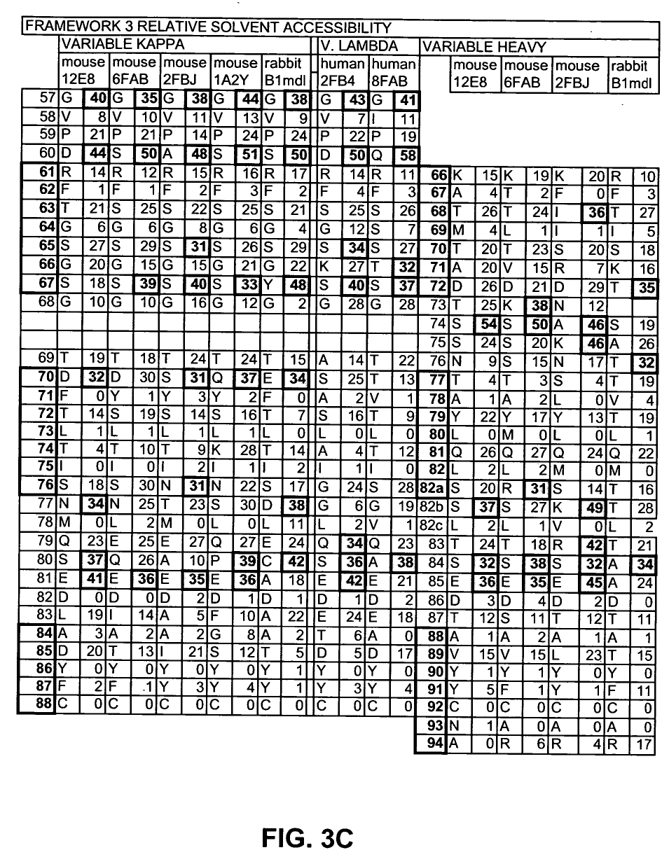

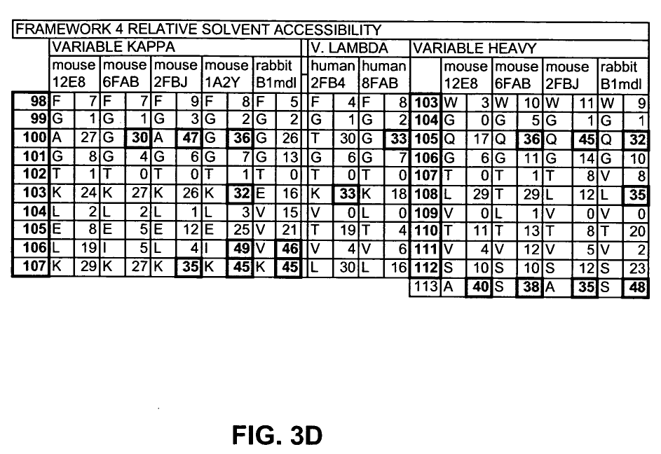

Exemplary modeling results are shown in FIGS. 3A-3D. Solvent accessible residues are indicated in shaded boxes.

The bottom loops are the A′-B, C—C′, C″-D, (C″ is in the CDR2 and so it is not shown in the figure), and E-F. A-A′ is not a loop though this region connects beta strands belonging to different sheets. All of these regions can be modeled by homology because the number of residues in them usually does not vary.

Steps 7-13

Once a model is made one can calculate the relative surface accessibility or all residues. For example the program Swiss Pdb Viewer can do these calculations. All surface residues, which are defined as having greater than 30% relative surface accessibility, can be humanized, unless they are in the CDRs or unless they contact the CDRs (contact distance is 5 A). Residues are therefore changed from the rabbit identity to the corresponding target sequence identity.

In other words, using the above methods, a) the surface residues of a rabbit antibody framework are determined. In some embodiments, these residues (shown in FIG. 3) are, for VH: 2, 3, 11, 13, 23, 26, 28, 41, 42, 72, 76, 84, 105, 108, 113 and for VK: 1, 3, 7, 9, 15, 18, 22, 40, 41, 42, 45, 57, 60, 67, 70, 77, 80, 106, 107; b) a list of the surface residues that are different to the corresponding surface residues of a similar non-rabbit host antibody are determined; c) D-E loop amino acids and CDR proximal amino acids are eliminated from the list.

As such, in many embodiments, nucleotide sequences encoding a subset of 2 or more, 4 or more, 6 or more, 8 or more or 10 or more residues of the above groups of amino acids are substituted to humanize/murinized a rabbit antibody.

As an option to confirm the prediction of surface residues in rabbit antibodies, a three-dimensional model can be constructed for the original and humanized or murinized antibodies. This can be done by simulating known methods for modeling murine and human antibodies, such as those described have been described (Martin et al Proc Natl Acad Sci 86: 9268-72 (1989); Martin et al. Methods Enzymol 203: 121-53 (1991). This method, called Combined Algorithm for Modeling Antibody Loops (CAMEL), is able to predict the backbone conformations of all six CDRs of the antibody binding site, as well as fitting together the framework regions. This method applies to human and murine antibodies equally well.

By substituting one or more nucleotides of a parent nucleic acid, as discussed above, a modified nucleic acid encoding the modified framework region is produced. As such, at least one nucleotide (i.e. about 1-5, about 6-10, about 11-15, about 16-20, about 21-30 about 31-40 or even more than about 50 nucleotides) of a parent framework region is altered to produce a nucleic acid encoding a framework region of a modified antibody. In most embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) is used to substitute, delete, and/or add appropriate nucleotides in the nucleic acid sequence encoding a parental antibody framework-coding sequence in order to create a modified framework-encoding sequence.

Several methods are known in the art for producing antibody-encoding nucleic acids, including those found in U.S. Pat. Nos. 6,180,370, 5,693,762, 4,816,397, 5,693,761 and 5,530,101. For example, site directed mutagenesis may be used to introduce/delete/substitute nucleic acid residues in the polynucleotide encoding a parental antibody framework region such that the mutagenized polynucleotide encodes a modified framework region. In other methods, PCR is used. One PCR method utilizes “overlapping extension PCR” (Hayashi et al., Biotechniques. 1994: 312, 314-5) to create modified rabbit V_{H} and V_{L} framework region-encoding sequences. In this method, the nucleic acid residue codons encoding the substituted/inserted/deleted amino acid residues in the modified polypeptide are engineered into PCR primers. Multiple overlapping PCR reactions using the parental nucleic acid sequence as a template generates a modified framework region. The product of many of these methods is a modified framework region. However, in performing these methods it is often possible to produce an amino acid that encodes an entire heavy or light chain variable domain, containing at least one modified rabbit framework sequence and a rabbit CDR.

In many embodiments, a modified variable domain-encoding nucleic acid is fused to a nucleic acid encoding an appropriate heavy chain, usually from a non-rabbit species such as humans or mouse. This is usually also accomplished using recombinant DNA technology techniques such as PCR, ligation, sub-cloning, etc. The sequences of human constant regions genes may be found in Kabat et al. ((1991) Sequences of Proteins of Immunological Interest, N.I.H. publication no. 91-3242) and human constant region-encoding sequences are readily available from known clones, e.g. from the A.T.C.C. The choice of isotype will be guided by the desired effector functions, such as complement fixation, or activity in antibody-dependent cellular cytotoxicity. In many embodiments, the constant region chosen is selected from IgG1, IgG3 and IgG4.

Antibody fragments, such as Fv, F(ab)₂ and Fab may be prepared by cleavage of the intact protein, e.g. by protease or chemical cleavage. Alternatively, the modified nucleic acid encodes an antibody fragment. For example, a chimeric gene encoding a portion of the F(ab)₂ fragment would include DNA sequences encoding the CH1 domain and hinge region of the H chain, followed by a translational stop codon to yield the truncated molecule.

Of course, modified framework encoding nucleic acids, modified variable domain-encoding nucleic acids, or even entire modified heavy or light chain-encoding nucleic acids or fragments thereof may be chemically synthesized.

In many embodiments the subject methods are performed by an algorithm by a computer or a computer system. In these embodiments, a user inputs at least the amino acid sequence of a framework region or a variable domain of a rabbit antibody into a graphical user interface, the computer performs the methods as described above, and outputs a modified rabbit framework or modified variable domain amino acid sequence or even a nucleotide sequence encoding a modified rabbit framework or modified variable domain a using an algorithm. Such programming is well within the abilities of one of skill in the art.

Programming according to the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above described methodology.

Nucleic Acids Encoding a Modified Rabbit Antibody

The invention further provides nucleic acids comprising a nucleotide sequence encoding a subject modified rabbit antibody, as well as portions thereof, including a light or heavy chain, a light or heavy chain variable domain, or a framework region of a light or heavy chain variable domain. Subject nucleic acids are produced by a subject method. In many embodiments, the nucleic acid also comprises a coding sequence for a constant domain. Since the genetic code is known, and the sequence of a heavy and/or and light chain variable domain framework regions can be determined for a modified rabbit antibody, the design and production of these nucleic acids is well within the skill of an artisan.

In most embodiments, the subject nucleic acids are substituted by at least one nucleotide of at least one codon of a framework region-encoding nucleic acid, such that the amino acid encoded by the codon encodes a corresponding amino in a donor framework region. In many embodiment, the nucleotides of two or more contiguous codons are substituted, whereas in other embodiments, the nucleotides of two or more discontiguous codons are substituted.

The subject nucleic acid segments may also contain restriction sites, multiple cloning sites, primer binding sites, ligatable ends, recombination sites etc., usually in order to facilitate the construction of nucleic acids encoding modified antibodies.

The DNA segments will typically further include an expression control DNA sequence operably linked to the humanized immunoglobulin coding sequences, including naturally-associated or heterologous promoter regions and terminators. In some embodiments, the expression control sequences will be eukaryotic promoter systems in vectors capable of transforming or transfecting eukaryotic host cells, but control sequences for prokaryotic hosts may also be used. Nucleic acids encoding a human immunoglobulin leader peptide (e.g. MGWSCIILFLVATAT, SEQ ID NO:27) may be engineered to allow the secretion of the antibody chains.

Vectors

The invention further provides vectors (also referred to as “constructs”) comprising a subject nucleic acid. In many embodiments of the invention, nucleic acid sequences encoding a modified rabbit antibody will be expressed in a host after the sequences have been operably linked to an expression control sequence, including, e.g. a promoter. The subject nucleic acids are also typically placed in an expression vector that can replicable in a host organisms either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like.

The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Host Cells

In most embodiments, the subject nucleic acids encoding a humanized monoclonal antibody are introduced directly into a host cell, and the cell incubated under conditions sufficient to induce expression of the encoded antibody.

Any cell suitable for expression of expression cassettes may be used as a host cell. For example, yeast, insect, plant, etc., cells. In many embodiments, a mammalian host cell line that does not ordinarily produce antibodies is used, examples of which are as follows: monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TR1 cells (Mather et al., Annals N.Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209.

Methods of introducing nucleic acids into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments lipofectamine and calcium mediated gene transfer technologies are used.

After the subject nucleic acids have been introduced into a cell, the cell is typically incubated, normally at 37° C., sometimes under selection, for a period of about 1-24 hours in order to allow for the expression of the antibody. In most embodiment, the antibody is typically secreted into the supernatant of the media in which the cell is growing in.

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule.

Once an antibody molecule of the invention has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Producing a Modified Rabbit Antibody

The present invention provides methods of producing a subject modified rabbit antibody. The methods generally involve culturing a subject host cell under suitable culture conditions and for a suitable period of time; and recovering the antibody.

A subject vector containing the DNA segments of interest (e.g., an expression cassette containing the heavy and light chain encoding sequences operably linked to expression control sequences such as a promoter and terminator) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (See, generally, Maniatis et al., Molecular Cloning: A Laboratory Manual, Cold Spring Harbor Press, (1982), which is incorporated herein by reference.)

Once the vector has been incorporated into the appropriate host, the host is maintained under conditions suitable for high level expression of the nucleotide sequences (eg. maintained under appropriate inducing conditions if an inducible promoter is used), and, as desired, the collection and purification of the modified antibodies or variants thereof will follow.

In many embodiments, the heavy chain and light vectors are co-transfected into the cell line and ELISA is used (Harlow et al,. Antibodies: A Laboratory Manual, First Edition (1988) Cold spring Harbor, N.Y.) to select stable cell lines that express both heavy and light chain genes, or, alternatively, the two chains are sequentially transfected into the cells and selected by different markers such as zeocin and hygromycin. As a third alternative, the heavy and light chain genes are transiently co-transfected to expression cells and the conditioned medium is used for antibody purification.

Once expressed, the whole antibodies, their dimers, individual light and heavy chains, or other immunoglobulin forms of the present invention, can be purified according to standard procedures of the art, including ammonium sulfate precipitation, affinity chromatography, size exclusion chromatography, gel electrophoresis or a combination of one or more of the foregoing, and the like (see, generally, R. Scopes, “Protein Purification”, Springer-Verlag, N.Y. (1982) and Harlow et al, supra).

In many embodiments, antibodies that are about 98% to 99% or even about 100% pure are required, however, and antibodies that are 90%-95%, 96%-98% about 50% pure or even unpurified will usually suffice.

Modified Rabbit Antibodies

The present invention provides modified rabbit antibodies made by the method of the invention.

In general, a modified rabbit antibody retains specificity for an antigen as compared to a parent antibody, has substantial affinity (e.g. at least 10⁷M⁻¹, at least 10⁸ M⁻¹, or at least 10⁹ M⁻¹ to 10¹⁰ M⁻¹ or more), and is less immunogenic in a non-rabbit host, as compared to a parent rabbit antibody. In many embodiments, the modified rabbit antibody contains at least one set of contiguous or non-contiguous amino acids from a non-rabbit antibody, such as a mouse or human antibody.

The level of immunogenicity of a modified rabbit antibody as compared to a parent rabbit antibody in a non-rabbit host may be determined by any of a number of means, including administering to a single non-rabbit host a formulation containing equimolar amounts of the two isolated antibodies and measuring the immune response of the non-rabbit host relative to each of the antibodies. Alternatively, the parent and modified antibodies are administered separately to different non-rabbit hosts and the immune response of the hosts are measured. One suitable method for measuring the immune response of the non-rabbit host relative to each of the antibodies is by ELISA (described in Ausubel, et al, Short Protocols in Molecular Biology, 3rd ed., Wiley & Sons, 1995, UNIT 11-4), where a suitable equal amount of each antibody is spotted into the wells of a microtitre plate, and the assay is performed polyclonal antiserum from the non-rabbit host. In most embodiments, a modified antibody is about 10% less immunogenic, about 20% less immunogenic, about 30% less immunogenic, about 40% less immunogenic, about 50% less immunogenic, about 60% less immunogenic, about 80% less immunogenic, about 90% less immunogenic or even about 95% less immunogenic than an unmodified parent antibody.

In many embodiments, the modified rabbit antibody is a basic antibody (i.e. a tetramer consisting of two identical pairs of antibody chains, each pair having one light and one heavy chain) or may be any variant of the basic antibody, such as a bifunctional antibody, a single chain antibody, Fab, Fv, F(ab′)₂ antibody etc, as long as it retains specificity, have substantial affinity and are less immunogenic in a non-rabbit host, as compared to a parent antibody.

A modified rabbit antibody may of course accommodate a level of amino acid variation, e.g. conservative amino acids substitutions, as long as they retain specificity, have substantial affinity and are less immunogenic in a non-rabbit host, as compared to a parent antibody.

Determining Binding Affinity of Modified Rabbit Antibodies

Once a modified antibody is expressed, it is usually tested for affinity using any known method, such as 1) competitive binding analysis using labeled (radiolabeled or fluorescent labeled) parent rabbit antibody, the modified antibody and an antigen recognized by the parent antibody; 2) surface plasmon resonance using e.g. BIACore instrumentation to provide the binding characteristics of an antibody. Using this method antigens are immobilized on solid phase chips and the binding of antibodies in liquid phase are measured in a real-time manner; and 3) flow cytometry, for example, by using fluorescent activated cell sorting (FACS) analysis to study antibody binding to cell surface antigens; 4) ELISA; 5) equibrilium dialysis, or FACS. In this FACS method both transfected cells and native cells expressing the antigen can be used to study antibody binding. Methods for measuring binding affinity are generally described in Harlow et al,. Antibodies: A Laboratory Manual, First Edition (1988) Cold spring Harbor, N.Y.; Ausubel, et al, Short Protocols in Molecular Biology, 3rd ed., Wiley & Sons, 1995).

If affinity analysis reveals a decrease in antibody binding for the modified antibody as compared to its parent antibody, framework “fine tuning” may be performed to increase the affinity. One method of doing this is to systematically change back each modified residues by site-directed mutagenesis. By expressing and analyzing these back mutant antibodies, one would predict the key residues that cannot be modified unless without decreasing affinity.

An alternative method to predict the residues that may need back-mutation is through molecular modeling. By comparing the 3-dimensional models of original and humanized or murinized antibody structure, any residues from the surface residues that are too close (e.g. <5 Angstroms) to the CDR residues, should be back-mutated to a residue of the rabbit or to a common residue for both species.

Utility

The invention provides methods for producing a modified rabbit antibody so that it is less immunogenic in a non-rabbit host than an unmodified parental rabbit antibody and modified rabbit antibodies made by these methods. These methods and compositions have several uses, many of which will be described below.

A modified rabbit antibody of the present invention find use in diagnostics, in antibody imaging, and in treating diseases susceptible to monoclonal antibody-based therapy. In particular, a humanized rabbit antibody may be used for passive immunization or the removal of unwanted cells or antigens, such as by complement mediated lysis or antibody mediated cytotoxicity (ADCC), all without substantial immune reactions (e.g., anaphylactic shock) associated with many prior antibodies. For example, the antibodies of the present invention may be used as a treatment for a disease where the surface of an unwanted cell specifically expresses a protein recognized the antibody (e.g. HER2) or the antibodies may be used to neutralize an undesirable toxin, irritant or pathogen. Humanized rabbit immunoglobulins are particularly useful for the treatment of many types of cancer, for example colon cancer, lung cancer, breast cancer prostate cancer, etc., where the cancers are associated with expression of a particular cellular marker. Since most, if not all, disease-related cells and pathogens have molecular markers that are potential targets for antibodies, many diseases are potential indications for humanized antibody drug. These include autoimmune diseases where a particular type of immune cells attack self-antigens, such as insulin-dependent diabetes mellitus, systemic lupus erythematosus, pernicious anemia, allergy and rheumatoid arthritis; transplantation related immune activation, such as graft rejection and graf-tvs-host disease; other immune system diseases such as septic shock; infectious diseases, such as viral infection or bacteria infection; cardiovascular diseases such as thrombosis and neurological diseases such as Alzeimer's disease.

Murinized rabbit antibodies find use as test therapies and imaging antibodies in mouse models of human diseases, such as mouse models correlate with the expression of a marker. As is known in the art, many of the disease examples listed above have corresponding mouse models. The molecular markers to which the antibody binds may reside in de-regulated “normal” cells such as immune cells (e.g. IL-2R, IL-4R being markers on these cells), endothelial cells (flt-1 and flk-1 being markers on these cells), etc. Alternatively, a marker may reside on or in a diseased cell such as a tumor cell or a pathogen cell, such as mdr-1 and p-glycoprotein in B16 mouse melanoma model.

Kits

Also provided by the subject invention are kits for practicing the subject methods, as described above. The subject kits at least include one or more of: a nucleic acid encoding of at least one framework sequence of a modified rabbit antibody that is less immunogenic in a non-rabbit species, an antibody encoded by such an nucleic acid, a vector containing the same, oligonucleotides primers for amplifying the same, nucleic acids encoding a constant domain for a non-rabbit species or oligonucleotides primers for the amplification thereof and a vector for expression of the modified rabbit antibody. Other optional components of the kit include: restriction enzymes, control primers and plasmids; buffers; etc. The nucleic acids of the kit may also have restrictions sites, multiple cloning sites, primer sites, etc to facilitate their ligation to non-rabbit antibody CDR-encoding nucleic acids. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Also provided by the subject invention is are kits including at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means for producing rabbit antibodies that are less immunogenic in a non-rabbit host than a parent antibody, or nucleotide sequences them.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Resurfacing of a Rabbit Monoclonal Antibody

The variable kappa and heavy chains of the rabbit anti-integrin beta6 monoclonal antibody B1 were PCR-cloned using the following PCR primers and conditions: several independent PCRs were done and the PCR products were sequenced.

Preparation of a Hybridoma Cell Suspension

spin 1 ml growing B1 cells 1100 RPM 5 min

wash with 1×PBS

count cells and adjust to 400,000 cells/ml

Preparation of RNA

Add 1 ul cells to 9 ul Buffer A on ice

Add 5 ul cold Buffer B

heat to 65° C. 1 min on heat block**** where?

cool gradually in Thermocycler 55° C. 45° C. 35° C. 23° C. Ice 30 sec 30 sec 30 sec 2 min

Add cold Buffer C −5 ul per tube

Incubate at 42° C. for 42 min

put back in Ice

Buffers A, B, C

Buffer A

2 ul DTT (0.1 M)

2 ul 5× first strand buffer

5 ul DEPC treated H2O

Buffer B

1.0 ul 0.1% NP40

1.0 ul First strand buffer

1.0 ul oligo dT

0.5 ul RNAseOUT 40 U/ml

1.5 ul DEPC treated H2O

Buffer C

1 ul 10 mM dNTP mix

1 ul 5× First strand buffer

1 ul Superscript RTII

2 ul DEPC treated H2O

PCR

Primer Concentration: 3 pmole/ul

2.50 ul 10× buffer

0.75 ul 50 mM MgCl2

3.00 ul primer 1

3.00 ul primer 2

0.50 ul 10 mM dNTP mix

0.25 ul Taq or other polymerase

10.00 ul Water

5.00 ul template

25.00 ul

94° C. 2 min

94° C. 30 sec|

57° C. 30 sec|×40 cycles

68° C. 25 sec|

68° C. 10 min

First round:

use for H chain: Primer 1+Primer 10

for L chain: Primer 12+Primer 19

Nested PCR

for H chain only: Primer 2+Primer 8

| | |
|---|---|
| >PRIMER 1 | |
| TCGCACTCAACACAGACGCTCACC | (SEQ ID NO:28) |
| >PRIMER 2 | |
| ATGGAGACTGGGCTGCGCTGGCTT | (SEQ ID NO:29) |
| >PRIMER 8 | |
| GCTCAGCGAGTAGAGGCCTGAGGAC | (SEQ ID NO:30) |
| >ODZPRIMER 10 | |
| TTGGGGGGAAGATGAAGACAGACGG | (SEQ ID NO:31) |
| >PRIMER 12 | |
| CAGTGCAGGCAGGACCCAGCATGG | (SEQ ID NO:32) |
| >PRIMER 19 | |
| GCCCTGGCAGGCGTCTCRCTCTA | (SEQ ID NO:33) |

The deduced protein sequences for the B1 antibody is as follows:

>B1 VK

(SEQ ID NO:34)

DIVMTQTPSSVSAAVGGTVTIKCQASDNIYSLLAWYQQKPGQPPKLLIYY
TSDLTSGVPSRFSGSGYGTEFTLTISDLECADAATYYCQSYHYSKSSTYV
NVFGGGTEVVVKG
>B1 VH

(SEQ ID NO:35)

QSLEESGGGLVKPGASLALTCKASGFSFSLSFYMCWVRQAPGKGLEWIAC
IYSGSSGSTYYASWAKGRFTISKTSATTVTLQMTTLTAADTATYFCARSA
SSTTFHYFNLWGQGTLVTVSS

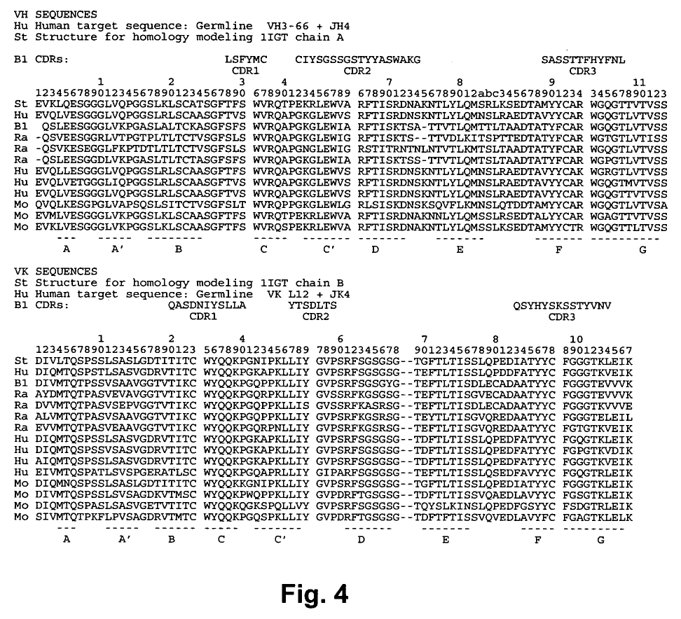

The sequences were aligned with the sequences shown in FIG. 2 in order to assign residue numbers, then a suitable human target sequence and a suitable structure sequence were also aligned. The alignments are show in FIG. 4, where the top three sequences for each chain are respectively the structure sequence for homology modeling, the desirable target human sequence, and the original B1 sequence. The remaining sequences are shown as in FIG. 1. For completion the CDRs of B1 are also shown above their insertion points.

A model of the rabbit B1 antibody VH/VK chains was made using the structure 1 IGT to thread the sequence as shown in the alignment. The program Swiss PDB Viewer for the original model as well as to calculate new loops for some of the CDRs and the VH D-E loop.

The relative surface accessibility was calculated (See FIG. 3 B1 mdl columns; Note CDR residues were already eliminated).

FIGS. 3(A-D) shows relative surface accessibility calculations for several high resolution structures as well as for a model of a rabbit IgG1, Kappa antibody (B1). The structure names and resolutions are, respectively: 12E8/1.9 Å, 6FAB/1.9 Å, 1A2Y/1.5 Å, 2FB4/1.9 Å, 8FAB/1.8 Å, and 2FBJ/1.95 Å. Only the frameworks are included and they are structurally aligned between the light and the heavy chains. That is, the beta strands, which are shown as bolded numbered residues on the left of each set of light or heavy chains, are aligned. Relative surface accessibility values greater than 30% are bolded. The figure demonstrates that surface positions are conserved (there is a positional consensus) and that a model of a rabbit antibody can be used to calculate which residues are on its surface. Sometimes a residue with a longer side chain can be exposed in one structure relative to another that has a shorter residue. For example position VH19 is an alanine in the rabbit antibody B1, which is not exposed by our calculations, whereas VH19 in all three structures is an exposed arginine. This makes sense because the alanine side chain (—CH3) is shorter and much less hydrophillic than the arginine side chain (CH2—CH2—CH2—NH—CN2H4+). This is an important point because it shows that often one needs to consider the positional consensus of exposed residues in order to make a decision about changing a particular residue.

Using the model, the exposed residues are:

VH: 2, 3, 11, 13, 23, 26, 28, 41, 42, 72, 76, 84, 105, 108, 113

VK: 1, 3, 7, 9, 15, 18, 22, 40, 41, 42, 45, 57, 60, 67, 70, 77, 80, 106, 107.

Exposed residues that are identical to corresponding residues in the human sequence (in 1IGT) are:

VH: 11, 26, 41, 42, 84, 105, 108, 113

VK: 1, 9, 15, 40, 41, 45, 57, 60, 70, 107.

If we eliminate these identical amino acids from the original set we are left with:

VH: 2, 3, 13, 23, 28, 72, 76

VK: 3, 7, 18, 22, 42, 67, 77, 80, 106.

Eliminating VH 76 because it is in the D-E loop, we get:

VH: 2, 3, 13, 23, 28, 72

VK: 3, 7, 18, 22, 42, 67, 77, 80, 106

Of these, residues that contact the CDRs are:

VH: 2, 28

VK: 3, 22, 67; and

Eliminating those from the previous set we are left with the final set of residues that can be changed such that most of the surface of the humanized antibody will “look” human, i.e. the rabbit antibody is resurfaced.

VH: 3, 13, 23, 72

VK: 7, 18, 42, 77, 80, 106

The final sequences of the resurfaced humanized rabbit chains are shown below with the changed residues in lower case:

>B1 VK

(SEQ ID NO:53)

DIVMTQsPSSVSAAVGGrVTIKCQASDNIYSLLAWYQQKPGkPPKLLIYY
TSDLTSGVPSRFSGSGYGTEFTLTISsLEpADAATYYCQSYHYSKSSTYV
NVFGGGTEVViK
>B1 VH

(SEQ ID NO:54)

QqLEESGGGLVqPGASLALTCaASGFSFSLSFYMCWVRQAPGKGLEWIAC
IYSGSSGSTYYASWAKGRFTISKdSATTVTLQMTTLTAADTATYFCARSA
SSTTFHYFNLWGQGTLVTVSS

Of course additional residues may be changed. For example the three ECA in the E-F loop may be changed to QPD because those residues are far from the CDRs and right at the tip of a loop. Of course, the invention does not preclude changing of additional residues as a calculated risk.

It is evident from the above results and discussion that the subject invention provides an important new means for resurfacing a rabbit monoclonal antibody. Specifically, the subject invention provides a system for identifying surface residues of a rabbit antibody, and altering them such that the surface of the antibody becomes more like that of a non-rabbit host antibody. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method for resurfacing a rabbit antibody, said method comprising:
(a) identifying a surface-exposed amino acid of a framework region of a parent rabbit antibody that differs from an amino acid at a corresponding position of a non-rabbit antibody by comparing the amino acid sequence of said framework region of said parent rabbit antibody to the amino acid sequence of said framework region of said non-rabbit antibody; and
(b) substituting said identified amino acid with an amino acid at said corresponding position of a non-rabbit antibody,
to resurface said rabbit antibody.

2. The method of claim 1, further comprising identifying amino acids of said framework region of a parent antibody that are proximal to a CDR and substituting only those amino acids that are not proximal to said CDR.

3. The method of claim 1, further comprising identifying amino acids in a D-E loop region of said parent antibody and substituting only those amino acids that are not in said D-E loop.

4. The method of claim 1, wherein said identifying step involves molecular modeling of said parent rabbit antibody or said non-rabbit antibody to identify said surface-exposed amino acids.

5. The method according to claim 1, wherein said identifying step (a) comprises identifying a plurality of amino acids and step (b) comprises substituting said plurality of amino acids.

6. The method according to claim 5, wherein said plurality of amino acids is at least two discontiguous amino acid.

7. The method according to claim 1, wherein said method is a method of humanizing a rabbit monoclonal antibody.

8. The method according to claim 1, wherein a modified rabbit antibody comprising a framework region having said substituted amino acid is less immunogenic in a non-rabbit host than said rabbit parent antibody.

9. The method according to claim 1, wherein said identifying step (a) comprises identifying amino acids that may be inserted into or deleted from said framework region of said parent rabbit antibody and said substituting step (b) comprises inserting into or deleting said amino acids from said nucleic acid sequence.

10. The method according to claim 1, wherein said rabbit antibody from a rabbit of known V_{H} allotype.

11. The nucleic acid according to claim 10, wherein said rabbit antibody is from a rabbit that is homozygous for a VH allotype.

12. The nucleic acid according to claim 19, wherein said rabbit is homozygous for an allotype chosen from V_{H}1-a1, V_{H}1-a2 and V_{H}1-a3.

13. The method according to claim 1, wherein said resurfaced antibody has a binding affinity of 10⁸ M⁻¹ or greater for a specific antigen.

14. A monoclonal antibody that has been resurfaced by the method set forth in claim 1.

15. A nucleic acid encoding the monoclonal antibody of claim 14.

16. A vector comprising the nucleic acid of claim 15.

17. A host cell comprising the vector according to claim 16.

18. A method of producing a modified rabbit antibody that is less immunogenic in a non-rabbit host as compared to its parent rabbit antibody, said method comprising:
incubating the host cell of claim 17 under conditions sufficient to produce said antibody; and
harvesting said antibody.

19. A computer-readable medium encoding instructions to direct a machine to perform the method of claim 1.

20. A kit for use in a computer, said kit comprising:
(a) a computer-readable medium according to claim 19; and
(b) instructions for operating said computer according to said programming.
